(19) United States
(12) Patent Application Publication
(10) Pub. No.: US 2002/0160436 A1
(43) Pub. Date: Oct. 31, 2002

(54) Method and apparatus for determining biologically useful field metrics associated with magnetic fields

(76) Inventors: Marko Markov, Chattanooga, TN (US); Floyd E. Bouldin, McMinnville, TN (US); C. Douglas Williams, Signal Mountain, TN (US); Rick R. Wascher, Rock Island, TN (US)

Correspondence Address: RICK R. WASCHER, EMF THERAPEUTICS, 111 LAW ROAD, McMINNVILLE, TN, 37110, US

(21) Appl. No.: 09/804,800

(22) Filed: Mar. 14, 2001

(51) Int. Cl. C12Q 1/48

(52) U.S. Cl. 435/15

(57) ABSTRACT

An apparatus and method for determining the biological amplitude and frequency windows of magnetic fields. The method for determining the biological window for the field metrics of a magnetic field is described with particular reference to a chemical system and method using myosin light chain kinase, calmodulin, and magnetic fields. The process is designed to calculate and measure the number of radioactive events (i.e., Cherenkov emissions) of a specimen or sample wherein such events are indicative of the relative biological effectiveness as will be described herein below.

A method for determining a biological window of a magnetic field comprising the steps of preparing a reaction solution containing at least the following components: MLC, MLCK, calmodulin, calcium ions, and radiolabeled ATP, and exposing the reaction solution to a magnetic field; removing the reaction mixture from the magnetic field and forming a specimen by placing a quantity of the solution onto a substrate; washing the specimen; placing the washed specimen in a suspension liquid and counting the number of radioactive events over a given time T.

### BACKGROUND OF THE INVENTION

1. Field of the Invention

This invention is directed to an apparatus capable of producing a magnetic field having an amplitude and frequency found beneficial for enhancing myosin phosphorylation which is known to be related to muscle activity and all eukaryotic cells in mammals.

The present invention is also directed to a method of quantitatively and qualitatively measuring and comparing the amplitude and/or the frequency of a subject magnetic field to a known relevant standard and thereby determining its relation to a biologically significant useful ranges of values (“windows”) confirmed by in vivo or in vitro experiments.

While the experimental apparatuses of the present invention may include virtually any configuration of a magnetic field generating device, apparatus or system, the preferred apparatus of the present invention is a magnetic field generating device of the type shown and generally described in U.S. Pat. No. 6,083,149, but also of a similar type as shown in 6,149,577 or 6,007,476, all of which are incorporated by reference as if fully set forth herein.

The method of the invention present is best described with particular reference to a bio-chemical system for cell free, in vitro myosin phosphorylation which effectively models in vivo myosin phosphorylation. The process includes the use of myosin light chains (“MLC”), myosin light chain kinase (“MLCK”), calmodulin, calcium ions, and radiolabeled ATP, in the presence of a magnetic field applied to sample specimens prepared from the aforementioned chemical system, then measuring the number of radioactive events emitted by a particular specimen during a prescribed observation period of time T.

2. Description of the Related Art

It is well established that electromagnetic fields (EMF) are capable of eliciting in vivo and in vitro effects from many biological systems. Selected low energy static permanent and time varying electromagnetic fields have been used for the past two decades to treat therapeutically resistant problems, mainly of the musculoskeletal system.

For purposes of obtaining a proper understanding of this inventive disclosure and the related art as it exists, one must consider the distinction between the meaning of the terms “pulsed” and “pulsating.” A “pulsed” field or signal includes a discrete “on/off” repetitive burst of signal emissions. The signal is comprised of a series of discrete allotments of signal components which when strung together do not have an interim continuous signal even if the interim non-burst signal were at or near zero. In this case, for example, if one were to consider water dripping from a faucet, the constant dripping would be a pulsed emission of water because of the lack of a continuous stream, even if the sizes of the drops differ from one to another.

A “pulsating” field can be described as having peaks (maximums and thus minimums) without an on/off condition. Applying the flowing water analogy to the pulsating field situation yields a constant and continuous flow of water with a variable velocity or volume (e.g., allowing the faucet to run and throttling the faucet open and closed to allow more or less water to flow therefrom), but never a zero flow condition. The distinction between pulsed and pulsating is particularly important as will become apparent because the field to which the apparatus of the present invention relates deals primarily with “pulsating” magnetic fields.

Magnetic field research also indicates that a proper assessment of the effect of an electromagnetic field exposure can only be done at the amplitude and spatial dosimetry of the induced electromagnetic field at the exact location of the target site. Of course, the frequency is considered to be held constant throughout. Therefore, while different maximums and minimums may be present in the actual target location (i.e., precise position where the data is being collected) or locations adjacent to it, it is particularly important to make all measurements at the location of the target even if the target covers an area larger than the measurement location (e.g., a target field). If the field metrics are not homogeneous throughout the target field, care must be taken to determine the actual target location where the data is being sampled in order that any replication or further verification by subsequent studies can be deemed reliable and useful.

Several magnetic field studies report the existence of “window” effects or resonance-type responses of biological systems to the amplitude and/or frequency metrics of the electromagnetic field. However, the whole range of environmental electromagnetic, electrostatic, and static magnetic fields, which could contaminate the experimental results need to be taken into account at the target site for proper measuring and thus replication/duplication of the experimental results.

During the past decade evidence has accumulated to show that contraction of smooth muscle like that of skeletal and cardiac muscles are directly calcium related. Calcium (Ca⁺²) is considered to be the main ion of interest in biomagnetics since the involvement of this ion is included within a number of critically important biochemical processes, including among other things for example nerve regeneration. Thus, the early modulation of calcium signaling by electromagnetic fields (“EMF”) is suggested to be a plausible candidate for activation of a number of biochemical reactions. EMF effects on calcium binding in tissue, for example, have been studied using cyclotron or quantum resonance EMF conditions.

Since all eukaryotic cells are known to contain actin, myosin, and other related proteins that are of primary importance in mobility of nonmuscular cells and contraction of cardiac, skeletal and smooth muscles, calcium ions appear to be essential in the first steps of transductive coupling of exogenous physical signals at the cell membrane and in the ensuing steps of calcium-dependent signaling to intracellular enzyme systems. Research shows the myosin light chain kinase (“MLCK”), from all muscle sources is dependent on Ca²⁺ as well as the specific calcium binding protein calmodulin. The active species contains kinase and calmodulin in a one-to-one molar ratio in the presence of Ca²⁺.

Calmodulin also plays a controlling role in many other important biochemical processes, such as cell proliferation, tumor promotion, oxocyte maturation, neutrophil activation, platelet function, Ca²⁺ membrane transport, insulin secretion, plant cell function, and others. Calmodulin regulation of enzyme activity has generally been found to require the presence of calcium ions. Calmodulin is capable of detecting micromolar concentrations of Ca²⁺ and once bound to calcium, calmodulin assume a more helical conformation to become the active species.

The crystal structure of calmodulin indicates that the protein consists of two globular domains, each containing two calcium binding sites connected by a continuous twenty-six residues of the alpha-helix type that separates the two globular domains. The COOH terminals bind Ca²⁺ with higher affinity than the NH₂ terminal sites (see FIG. 2). Both terminal pairs are separated by a single solvent-exposed “central-helix” which yields an overall dumbbell shape to the protein. The binding in the COOH-terminals is largely responsible for the Ca²⁺ induced structural changes.

Phosphorylation, sometimes called chemiosmosis is defined as a phenomenon in which an energy dependent transfer of protons or electrons across an energy transducing membrane generates or augments a transmembrane proton gradient whose inherent energy can be used for chemical, osmotic or mechanical work. It is known the Ca²⁺-calmodulin-dependent phosphorylation of myosin occurs in the following manner: Ca²⁺ binds to calmodulin, causing a conformational change in calmodulin; the calcium/calmodulin complex then interacts with the inactive catalytic subunit of MLCK to form a catalytically active holoenzyme complex; the kinase proceeds to phosphorylate MLC. Calcium at micromolar concentrations is assumed to be obligatory for complex formation. MLCK is the protein that preferentially catalyzes the phosphorylation of a specific light chain subunit of myosin by transfer of the gamma-phosphate of ATP to a serine residue on a specific class of myosin light chains.

### SUMMARY OF THE INVENTION

Studies in vitro with purified smooth muscle and nonmuscle myosin have shown that the phosphorylation of myosin light chain kinase (“MLC”) affects polymerization and stabilization of myosin filaments. It was shown that in smooth muscle cells phosphorylation leads to an increase in actomyosin ATPase activity, while in skeletal muscle MLC phosphorylation correlates with potentiated twitch tension after repetitive stimulation and increases the rate at which myosin crossbridges move into the force generating state. Ca²⁺ binding protein, calmodulin (CaM), plays the most important role in the activation of myosin light chain kinase (MLCK).

Studies on molecular and subcellular mechanisms of Ca²⁺-CaM and Ca²⁺-CaM-enzyme interactions revealed calcium ion as an important regulator of contractile protein interactions. Accordingly, the myosin phosphorylation model is particularly useful when studying magnetic field effects in biological systems because the myosin phosphorylation model reacts to the field metrics in much the same manner as certain mammalian tissue would likely also react to the same field metrics.

The inventive method for determining the relative biological effectiveness of a magnetic field is a process of measuring the activity of a mono-phosphate by-product of the radioactively labeled ATP (i.e., ATP tagged with radioactive phosphorous ions) with respect to cell free myosin phosphorylation of a specimen subjected to a magnetic field. Specifically, the inventive method is therefore summarized with particular reference to a chemical system and method using MLC, MLCK, calmodulin, calcium ions, ATP and magnetic fields.

The process is designed to measure the number of radioactive events of a specimen or sample which is indicative of the relative biological effectiveness of the subject field via repetitive experiments fluctuating the field metrics. The measurable cell free myosin phosphorylation values are collected and compared to determine any correlation between them including the static [or constant] magnetic field values of the permanent magnets and the values associated with the preferred embodiment of the apparatus. While other chemical systems may exist and thus while the claims may include limitations disclosing to the preferred embodiment of the chemical system, they also are not so limited.

The confirmation of the relative biological effectiveness of a magnetic field having the preferred amplitude and frequency metrics of the present invention was done by comparing cell free myosin phosphorylation data to previously collected in vitro biological data obtained from prior animal research using magnetic fields of the type associated with the present invention. The cell free myosin phosphorylation data confirmed the biologically useful field metrics used in the animal studies and this independent verification renders the cell free myosin phosphorylation technique highly useful as an economical, time and resource efficient way of determining the relative biological effect in mammals of certain magnetic field applications.

To use the cell free myosin phosphorylation technique as an indicator of relative biological effectiveness generally includes the following steps: preparing a calcium calmodulin solution, exposing the solution to a magnetic field under certain conditions such as temperature and exposure time, stopping the reaction, preparing a sample specimen, and counting a number of radioactive events associated with the exposed specimen over a time T.

The radioactive events are preferably measured by a liquid scintillation counter which measures the Cherenkov emissions by a volumetric proportion to the sample being measured. The greater the cell free myosin phosphorylation activity (ie., greater number of Cherenkov counts) determines the preferred field metrics associated with the magnetic field. In vivo experimentation in animals can be and was used to determine and confirm the preferred field metrics of the cell free myosin phosphorylation technique performed in vitro.

For example, with respect to biologically useful field metric windows the specimens were exposed to a magnetic field between 5 and 55 milliTesla for both biological amplitude windows, between 5 and 25 milliTesla (e.g., 15 mT) for the first biological amplitude window and between 30 and 55 milliTesla (e.g., 45-50 mT) for the second biological amplitude window. The biological frequency window is determined to be the number of hertz, but more properly the number of pulses per second as will be described herein below which was found to be equal to twice the frequency of the commercially available power supply.

Where any reference specimen (e.g., a specimen subjected to any magnetic field) is also analyzed along with a target, the method is as follows: preparing a calcium calmodulin solution; exposing a first amount of solution to a first magnetic field and a second amount of solution to a second magnetic field having a magnitude corresponding to a biological amplitude window. The preferred embodiment of the inventive method uses a first and second solution having the same concentration of the solution components (e.g., a first and second solution drawn from a common source thereof). After exposure of preferably five (5) minutes the reaction is stopped, and the solution is used to create samples from which the number of radioactive events associated with the reference and target specimen are measured/counted. The same process is used for the reference and target specimen and the same solution is also used. The number of radioactive events of the target are compared to the number of radioactive events or field metrics of the reference as a means of comparison. For example, rather than Cherenkov emissions, the reference specimens comparative data can be extracted from previously known values such as those associated with the field metrics of animal studies, prior experiments, etc.

The present invention may be summarized in a variety of ways, one of which is the following: a method for determining a biological window of a magnetic field comprising the steps of preparing a reaction solution containing at least the following components: MLC, MLCK, calmodulin, calcium ions, and radiolabeled ATP; exposing the reaction solution to a magnetic field; removing the reaction mixture from the magnetic field and forming a specimen by placing a quantity of the solution onto a substrate; washing the specimen; and placing the washed specimen in a suspension liquid and counting the number of radioactive events over a given time T.

The present invention may also be summarized as follows: a method for determining the relative biological effectiveness of a magnetic field using cell free myosin phosphorylation comprising the steps of preparing a reaction solution containing at least the following components: MLC, MLCK, calmodulin, calcium ions, and radiolabeled ATP; exposing a first volume of the reaction solution to a first magnetic field; exposing a second volume of the reaction solution to a second magnetic field; removing the reaction mixture from the first magnetic field and forming a first specimen by placing a quantity of the first volume of solution onto a substrate; removing the reaction mixture from the second magnetic field and forming a second specimen by placing a quantity of the second volume of solution onto a substrate; washing the first specimen; washing the second specimen; placing the washed first specimen in a suspension and counting the number of radioactive events over a given time T; and placing the washed second specimen in a suspension and counting the number of radioactive events over a given time T.

A preferred method also includes exposing the first specimen to the first magnetic field and exposing the second specimen to the second magnetic field both for a period of time within the linear portion of the time dependence curve of myosin phosphorylation rate; and/or exposing the first specimen to the first magnetic field for a period within the range of time between 2 and 6 minutes but preferably 5 minutes.

A preferred method also includes creating a first or second magnetic field prior to exposure such that the first or second magnetic field has a frequency of 80 to 180 pulses per second, but preferably 100 or 120 pulses per second. Similarly, a preferred method also includes creating a first or second magnetic field prior to exposure such that the first or second magnetic field has an amplitude between 5 and 55 milliTesla, but preferably 15-20 mT or 45-50 mT.

The preferred apparatus is a coil assembly including at least one electrical conductor; and a source of electric current applied to the length of electrical conductor to create a magnetic field having an amplitude within a known biological magnetic field metric window within the interior of the coil Further, the preferred apparatus includes a frame defining a coil assembly interior when the coil is wrapped about the frame, a central passageway extending through the frame, and a useful magnetic field frequency in pulses per second which is double the frequency of the input voltage and corresponding current obtained from a readily available commercial electric power supply. A rectifier is preferred for doubling the frequency of the input voltage and corresponding electric current.

The coil assembly is configured to create a magnetic field having a frequency of 80 to 180 pulses per second, and 5 and 55 mT. The preferred frequency is 100 to 120 pulses per second and the preferred amplitude is 15-20 mT and 45-50 mT.

The apparatuses used to produce the magnetic field to which the target samples or specimens were subjected in the present invention included, but not limited to, the following: a magnetic field created by a permanent magnet; a pair of spaced apart magnets with opposing polar faces toward one another; and, a magnetic field generating device of the type shown and described in U.S. Pat. No. 6,083,149, but also of a similar type as shown in U.S. Pat. No. 6,149,577 or U.S. Pat. No. 6,007,476, all of which are incorporated by reference as if fully set forth herein.

The definition of the term “hertz” or the abbreviation “Hz” is well known. The definition of the phrase “pulses per second”, is similar and related to hertz and refers to the frequent repetitive occurrences of an amplitude maximum value. The common definition of the term hertz defines a wave form which alternates between a maximum or positive value and an identical minimum but negative value. The number of times this repetitive period is reproduced in one second is the frequency in hertz (Hz).

Therefore, while the term hertz might be useful for familiarity and to maintain consistency with the terminology with U.S. Pat. Nos. 6,083,149, 6,149,577 and 6,007,476, the term hertz is more properly replaced here with pulses per second because the frequency data and disclosure of the present invention shall be referred to in the context of a frequency output rather than the input signal like the aforementioned patents use.

That is, a typical 50 or 60 hertz frequency of an input voltage and corresponding current supply has 50 or 60 maximum values and 50 or 60 minimum values alternating together to form a repetitive period in which one maximum and one minimum create the input wave form for the alternating current supply. With the respect to the present invention a 100 or 120 hertz is more properly referred to as pulse per second on the output side because like the aforementioned patents, the wave form is changed to provide magnetic field metrics found biologically useful as changed. Thus, the inventive field may use the same 50 or 60 hertz input frequency and have the conventional current wave form with the period containing a single maximum and a single minimum value, the inventive apparatus yields 100 or 120 pulses per second (i.e., depending upon whether the input is 50 or 60 hertz for example) of all maximum values, such that the 50 or 60 times the value reaches a maximum is added to the 50 or 60 times the value would ordinary reach a minimum but for the upward inversion (i.e., above the zero reference line) or “absolute value” of the minimum negative values.

This “inversion”, accomplished by the bridge rectifier and full wave rectification transforms a 50 and 60 hertz signal to a 100 and 120 pulse per second signal respectively. The doubling or wave pair arrangement of above the zero reference line when viewing the wave form yields a pulsating magnetic field having a select number of pulses per second which is the absolute value of two times the input frequency of the supply current. The given amplitude as will be shown herein is the preferred IT amplitude of the amplitude windows. The use of the term hertz therefore is acceptable, but is better defined here as pulse per second because of the field windows defined and discussed herein.

The preferred embodiment of the apparatus comprises a tightly wound coil of continuous wire wrapped about a non-conductive frame, preferably made of phenolic resin impregnated spun glass fibers, in a manner similar to winding thread around a spool. A current is passed through the coil in one of two directions “+” positive or “−” negative, (i.e., to the right or to the left). The current carrying coil produces a magnetic field. The number of coil wire turns may vary.

Various embodiments of the present inventive apparatus incorporate devices using between fifty (50) and one thousand six hundred (1600) turns of copper wire were used. The coils themselves may be a single coil or multiple individual coils in a stacked or adjacent relationship where the total number of coil windings is counted. It is important to note the number of windings is not believed to be critical so long as the preferred amplitude and frequency can be generated from the coil. Efficiency and input power concerns generally help dictate the number of coil turns due to the relative cost of the electrical supply to the machine and material to create it.

A preferred power supply is a variac type transformer or signal generator capable of delivering sufficient current, depending upon the number of coil windings as mentioned, to generate up to 55 milliTesla—the outer limit of the investigated treatment window with respect to the amplitude metric. The corresponding voltage to achieve the appropriate current again depends upon the number of turns of wire used to form the coil since the input power supply is a 110 or 220 volt (i.e., 110 V or 220 V) 60 cycle (hertz or Hz) supply voltage for studies done in the United States and 50 Hz input supply for studies done outside of the United States of America. For example in one U.S. study ((“Study—EXAMPLE FOUR” of U.S. Pat. No. 6,083,149) (incorporated by reference as if fully set forth herein)), 7.5 amps current translated to 15mT output of the device which was found to be a part of the preferred amplitude window values.

Other supply voltages are contemplated depending upon the nature of the electrical distribution of the locality, or non-standard supply voltage present in a system in which the apparatus is used. The AC input voltage applied to the coil may be passed through a voltage regulating device for changing (i.e., increasing or decreasing) the voltage amplitude as desired by the operator depending upon the application. In the alternative, where fixed voltages are used or desired, for example in the coil assembly embodiments having a large number of windings a step up transformer may be used to provide a preselected steady state voltage (i.e., the working voltage from the variac type device) emerging therefrom The working voltage is directed to a rectifier to convert the AC input signal to rectified signal. The AC voltage is preferably rectified by a full-wave rectifier set to achieve the doubling in pulses per second (i.e., inverted doubling of the frequency associated with the supply wave form).

The rectifier converts the input frequency of either 60 pulses per second (i.e., half wave rectification by eliminating the minimum values of the input) or 120 pulses per second (i.e., full wave rectification by flipping the minimum values upward) depending upon the rectifier setup. Similarly, where fifty (50) pulse per second voltage is used as the AC supply, the resulting pulse per second frequency is either 50 or 100 pulses per second depending upon the rectification. The harmonics of 50 and 60 pulses, or 100 and 120 pulses are also believed to be useful to achieve the desired result, or they may be filtered to eliminate them and their associated affects.

In the United States of America the power supply is a 60 hertz (i.e., pulse per second) supply and the apparatus of the present invention incorporates a rectifier as one way of achieving the 120 pulse per second frequency window confirmed by the inventive cell myosin phosphorylation method disclosed herein and supported by animal data previously collected in “Study 4—EXAMPLE FOUR” of U.S. Pat. No. 6,083,149 (incorporated by reference as if fully set forth herein).

The present invention may also be summarized as an apparatus for providing a magnetic field having useful biological effects, comprising: a magnetic field amplitude of 15-20 mT and 45-50 mT; and a useful magnetic field pulse frequency equal to the absolute value of the number of maximum values of the supply frequency which translates into a number of pulses per second which is twice the frequency of the input current obtained from a readily available commercial electric power supply. The preferred field frequency is preferably 120 and 100 pulses per second.

The preferred apparatus further comprises a coil assembly including at least one electrical conductor; and a source of electric current applied to the length of electrical conductor to create a magnetic field within the interior of the coil A frame defines the preferred coil assembly interior when the coil is wrapped about the frame, and a central passageway extending through the frame. The preferred apparatus also includes a rectifier for doubling the frequency of the input electric current.

### BRIEF DESCRIPTION OF THE DRAWINGS

FIG. 1 is a graphical process flow chart corresponding to a chemical process associated with the method of the present invention;

FIG. 2 is a representative drawing of calmodulin and the calcium binding sites inherent within the chain;

FIG. 3A is a representative process flow chart corresponding to the myosin phosphorylation chemical process associated with muscle and other tissue in vivo;

FIG. 3B is a representative process flow chart corresponding to the myosin phosphorylation chemical process associated with the method of the present invention in vitro;

FIG. 3C is a graph illustrating the linear portion of the time dependence curve of myosin phosphorylation rate from which the exposure times are preferably selected;

FIG. 4. is a graph illustrating the relative dependence of CD31 staining on an applied magnetic field produced with a preferred amplitude and frequency illustrating the relative effectiveness of a variety of magnetic field amplitudes indicating relative biological effectiveness;

FIG. 5. is a graph illustrating a decrease in CD31 staining response of sample tissue as compared to a control tissue sample illustrating the relative effectiveness of a variety of magnetic field amplitudes indicating relative biological effectiveness;

FIG. 6 is a graph illustrating myosin phosphorylation as a function of applied magnetic field in order to illustrate the existence of biological magnetic field amplitude windows;

FIG. 7 is a graph illustrating myosin phosphorylation as a function of applied electromagnetic field strength and static magnetic field strength to further illustrate the existence of biological magnetic field amplitude windows and the comparative relative effectiveness of each field configuration;

FIGS. 8 and 9 are bar graphs illustrating the existence of a frequency dependant biological window as confirmed by the inventive method used herein; and

FIGS. 10-15 are graphs illustrating the wave forms of some of the various wave forms associated with the present invention and further illustrating the inverted doubling of the frequency to a pulse per second output signal.

### DETAILED DESCRIPTION OF THE PREFERRED EMBODIMENTS

Myosin light chain (MLC) phosphorylation is required for ATPase activity which is known to accompany smooth muscle contraction. Experiments were performed by using myosin light chains and myosin light chain kinase isolated from turkey gizzard obtained from M. Ikebe (University of Massachusetts, USA).

The reaction mixture consisted of a basic solution containing 40 mM Hepes buffer, pH 7.0; 0.5 mM magnesium acetate; 1 mg/ml bovine serum albumin; 0.1% (w/v) Tween 80; and 1 mM EGTA. A U solution containing 2.5 μM Ca²⁺, 70 nM CaM, 160 nM MLC and 2 nM MLCK was added to the basic solution to form the final reaction mixture. Each solution is prepared daily and considered “fresh”. The low MLC/MLCK ratio was chosen to obtain a linear time behavior. The above listed concentrations provide reproducible enzyme activities and minimize eventual pipetting errors.

As mentioned, the reaction mixture was freshly prepared daily for each series of experiments and was aliquoted (by 100 μL portions) into 1.5 ml Eppendorf tubes. All Eppendorf tubes containing the reaction mixture were kept at 0° C. then transferred to a plastic chamber having an interior water filled reservoir surrounded by an outer chamber wall which serves as a flowing water jacket enabling a constant perfusion of water prewarmed to (37.0±0.1)° C. by passage through a Fisher Isotemp 1006S heat exchanger to maintain a constant temperature of the water within the reservoir. Temperature was monitored to ±0.1° C. with a Fisher Traceble thermometer immersed in the interior water filled reservoir of the chamber during all experiments. This chamber was placed in the magnetic field to be evaluated.

The reaction was initiated by adding 2.5 μM ³²P ATP(2000-6600 cpm/pmol) to the reaction medium, and was stopped with Laemmli Sample Buffer (LSB) solution (100 μL), which contains 30 μM EDTA after five (5) minutes of exposure. After applying LSB stopping solution, the reaction suspension is thoroughly mixed and then aliquoted on 2×14 cm 3 MM filter paper. There are preferably 6 spots or specimens and each is 2×2 cm to assure adequate washing. Then the pieces of filter paper are placed in a 1000 ml beaker which contains 500 ml of 30% solution of TCA. The samples are allowed to be in the beaker for 20 min and then they are transfer in a second beaker containing 500 ml 15% solution of TCA. The third, and final washing is equivalent to the second one. After completing the washing procedure the individual 2×2 cm squares were placed in scintillation vials containing 20 ml distilled water to be counted for radioactively labeled monophosphate as an indicator of reaction efficiency. The washing step isolates the radiolabeled mono-phosphate released by ATP for counting Cherenkov emissions.

At least five blank samples were counted in each experiment. Blanks consisted of the total assay mixture minus Calcium ions. It is known that the Cherenkov counts for blanks are identical when any one of the active components (Ca²⁺, CaM, MLC, MLCK or ATP) is not included in or isolated from the solution, hence stopping the reaction. When blank counts were lower than 300 cpm the experiment was not accepted. During the experimentation, several control specimens which were used for experimentation were also prepared and carefully recorded.

Phosphorylation was evaluated using a Beckman liquid scintillation counter which counted the Cherenkov emission due to ³²P incorporation into myosin light chains. All experiments were repeated a minimum of five (5) times. A Student's paired t-test was performed for each time and exposure condition. Significance was accepted at p<0.05.

The preferred apparatus of the magnetic field exposure system for delivering the magnetic field consisted of a magnetic field generating device invented and produced by EMF Therapeutics, Inc. of Chattanooga, Tenn. and described in U.S. Pat. No. 6,083,149 and also of the type disclosed in U.S. Pat. Nos. 6,007,476 or 6,149,577 all of which are incorporated by reference as if fully set forth herein (hereinafter the “EMF Therapeutics Devices”). The magnetic field exposure system, therefore, consists of an ellipsoidal coil having an interior passageway capable of generating a pulsating magnetic field with a frequency of twice the incoming supply frequency (e.g., 60 Hz supply voltage yields 120 Hz/pulse per second field frequency, and 50 Hz supply voltage yields 100 Hz/pulse per second field frequency).

Magnetic field effects on Ca²⁺-calmodulin dependent myosin phosphorylation occurs for Ca²⁺-depleted conditions during the nonequilibrium phase of the reaction. For these conditions, kinetics favor the bound state according to kₒₙ/k_{off}. 10²-10³, the instantaneous exchange reaction rate, v(t), is dependent upon the instantaneous free Ca²⁺, and phosphorylation increases for increasing Ca²⁺(t). Ca²⁺(t) is proportional to the ratio of the time the ion is free (unbound) to the time bound, computed over a time interval sufficiently large in comparison with the mean time for interwell hopping:

$$[ {Ca}^{2 +} ( t ) ] = ρ {t}_{\frac{{}_{free}}{{t}_{bound}}} ,$$

where ρ is a proportionality constant. Therefore v(t), which is proportional to the concentrations of free ions and CaM in the linear phase of the reaction is:

$$v ( t ) ∝ [ {Ca}^{2 +} {CaM ( {H}_{2} O )}_{j} ] + [ {kH}_{2} O ] ← {t}_{\frac{{}_{free}}{{t}_{bound}}} + [ {CaM ( {H}_{2} O )}_{m} ]$$

$$ρ$$

Changes in applied fields that cause the ion dynamics to favor the free state will yield an increase in Ca²⁺(t), causing an increase in instantaneous reaction rate, i.e., an increase in net bound Ca².

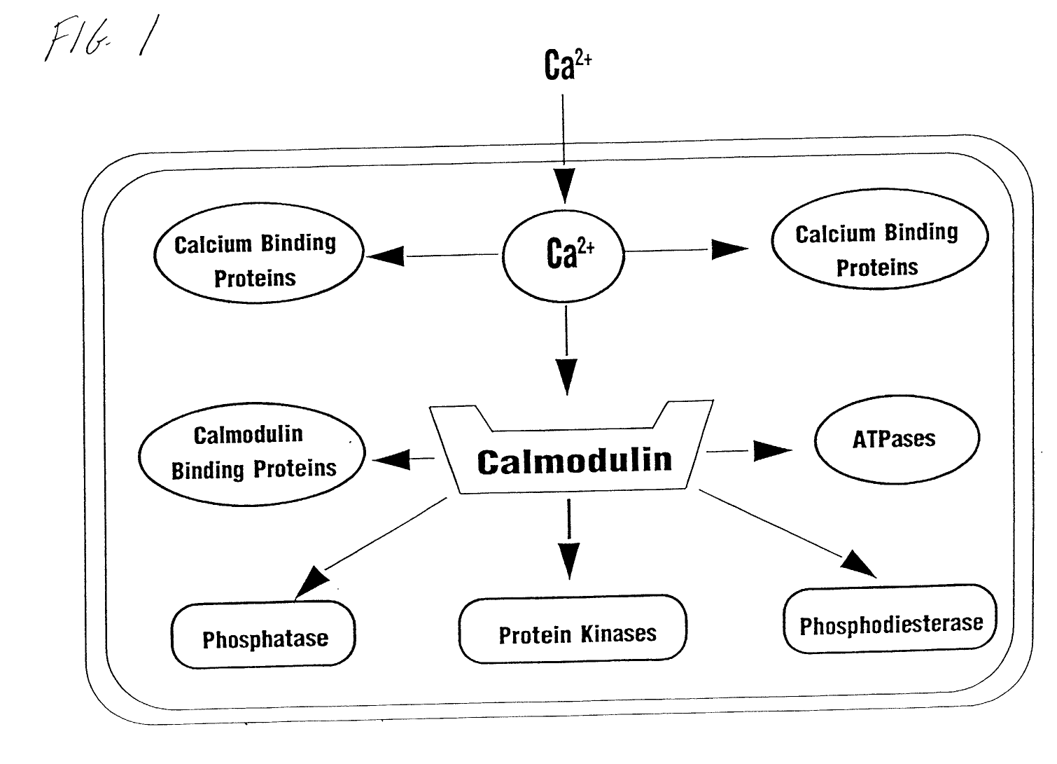

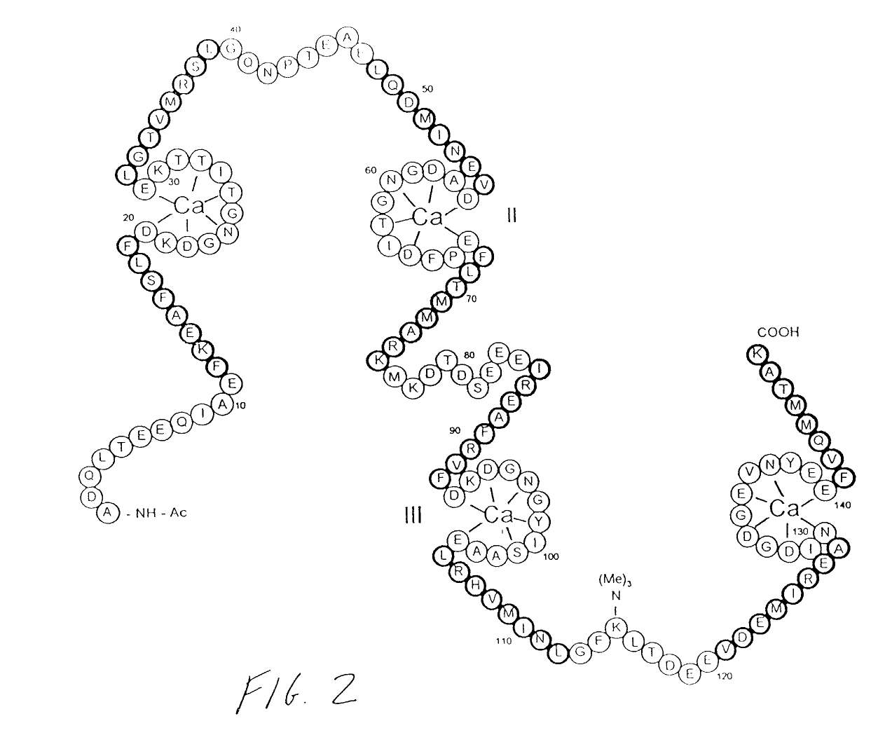

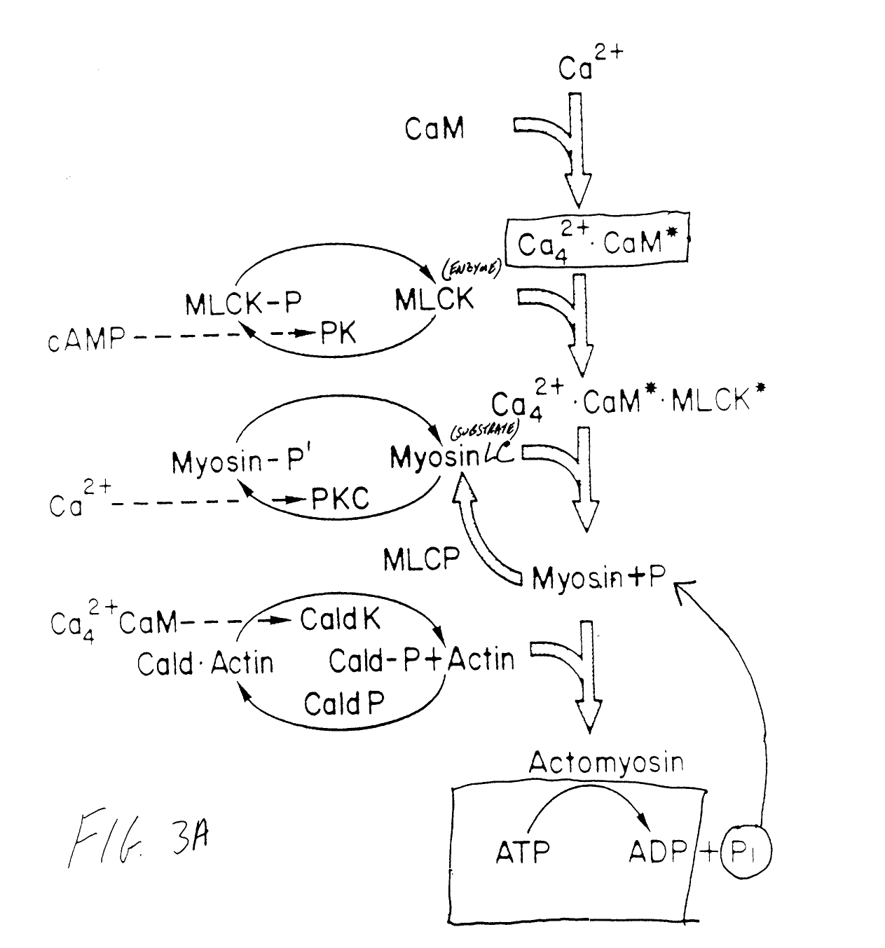

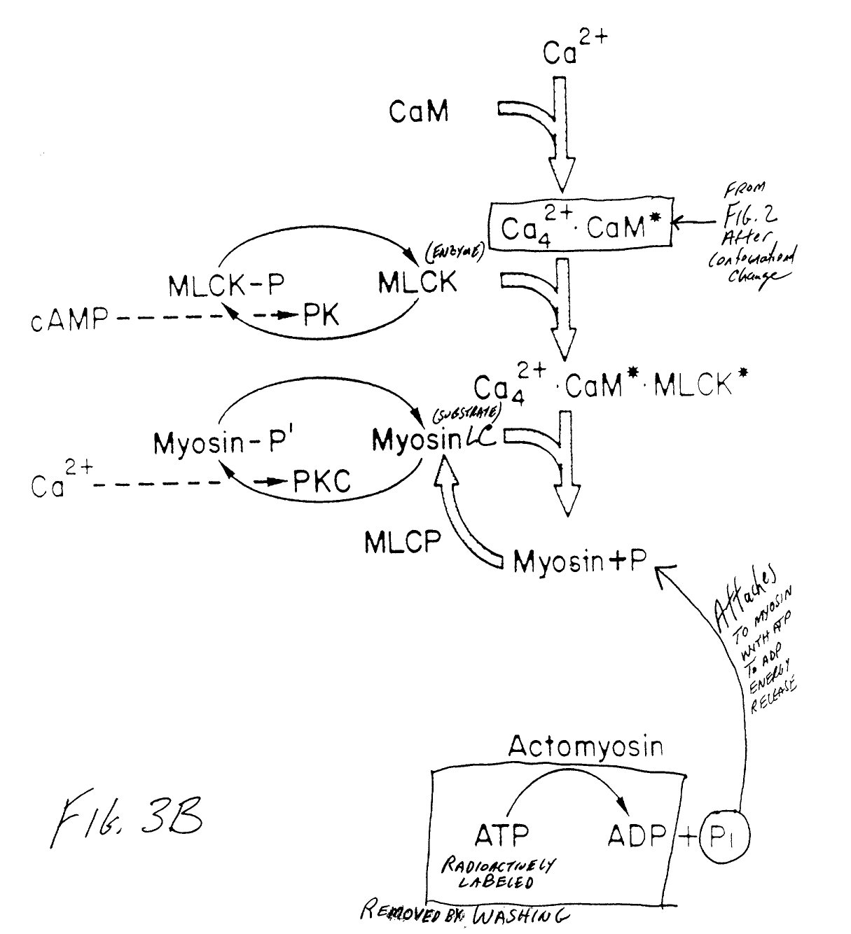

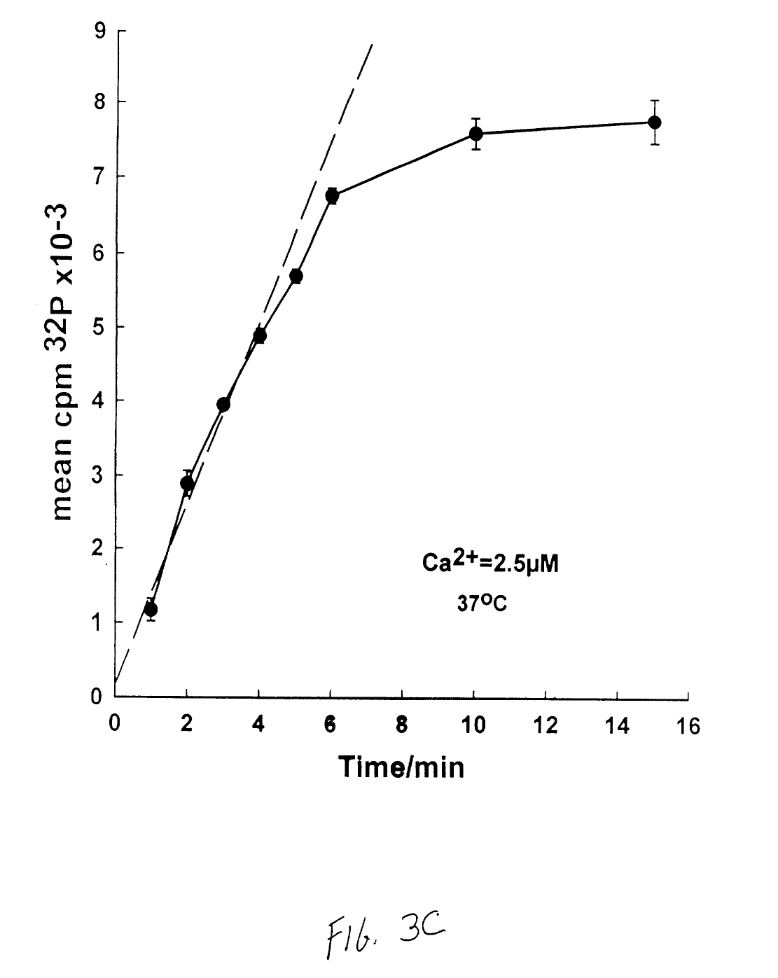

The critical step is the binding of Ca²⁺-CaM complex to MLCK which activates the kinase. With reference to FIGS. 1, 2, 3A, 3B and 3C, the structure of calcium/calmodulin and myosin phosphorylation reaction is discussed. The calcium/calmodulin is a dumbbell shaped complex with an overall length of approximately 65 angstroms and consists of two globular domains. Each domain contains two Ca²⁺ binding sites of the helix loop helix type connected by a long rigid central helix. In the absence of bound calmodulin, myosin light chain kinase of turkey gizzard is phosphorylated at the two sites A and B. The extent of phosphorylation was estimated by LCB liquid scintillation counter which counted gamma P incorporated into myosin light chain. FIG. 3C illustrates the preferred reaction time is best chosen in the linear portion of the time dependence curve of myosin phosphorylation rate where the reaction is predictable.

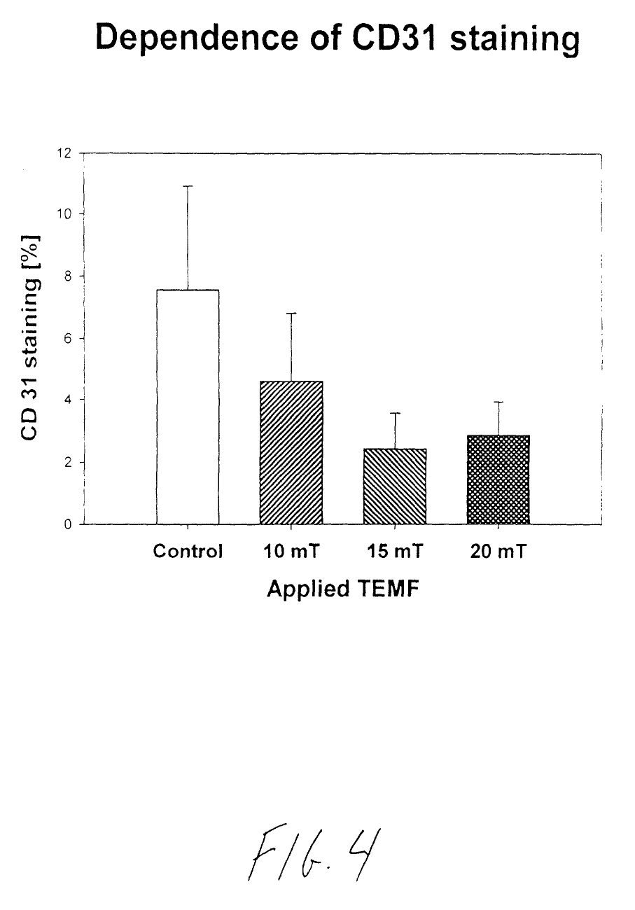

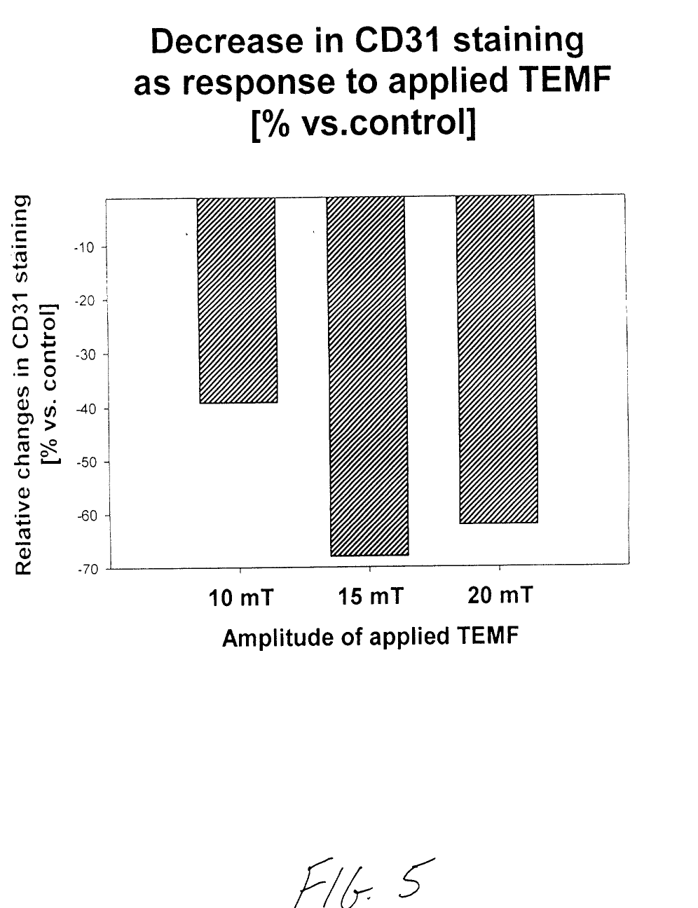

FIGS. 4 and 5 graphically illustrate the effect of exposure to a pulsating field from the EMF Therapeutics Devices at various amplitudes. The results shown in the Figures taken from the animal studies disclosed in the “Study 4—EXAMPLE FOUR” of U.S. Pat. No. 6,083,149 (incorporated by reference as if fully set forth herein) demonstrates a 7.5 Amp current through the EMF Therapeutics Device (Le., yielding a preferred 15 mT amplitude) produced the greatest beneficial biological results in the mammals studied.

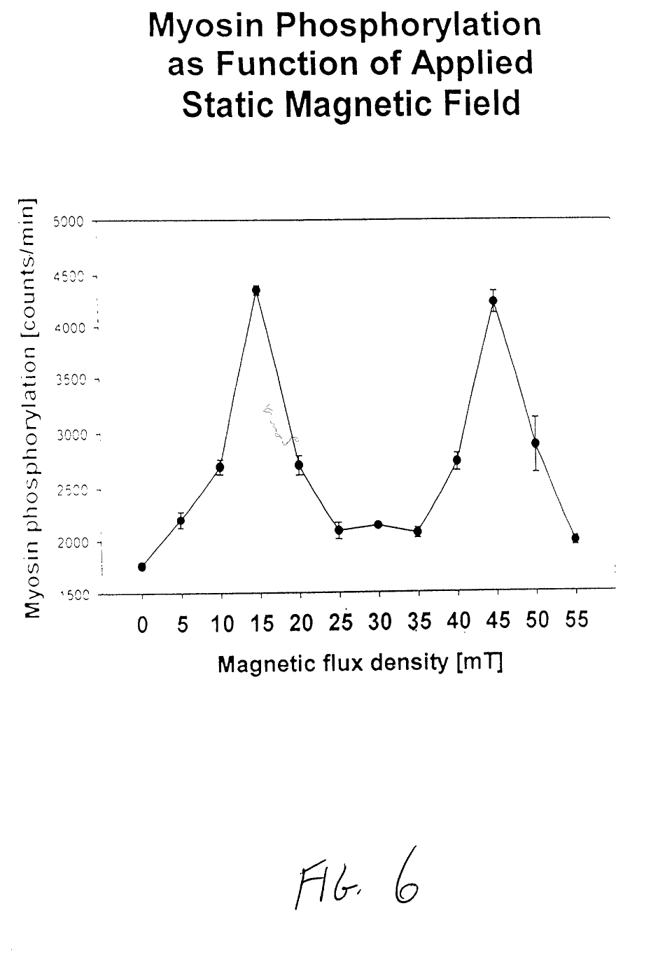

The cell free myosin phosphorylation technique of the present invention was used in conjunction with a static/constant magnetic field generated by a single permanent magnet separated from the target which gives rise to gradient fields, or a pair of permanent plate-like magnets spaced apart from one another with opposing poles facing each other in order to establish a homogeneous magnetic field (both of which can be referred to collectively and individually as “constant magnetic fields”). The constant magnetic fields as measured by the myosin phosphorylation model defined herein and shown in FIG. 6 demonstrate and confirm the existence of magnetic field biological amplitude windows for these fields.

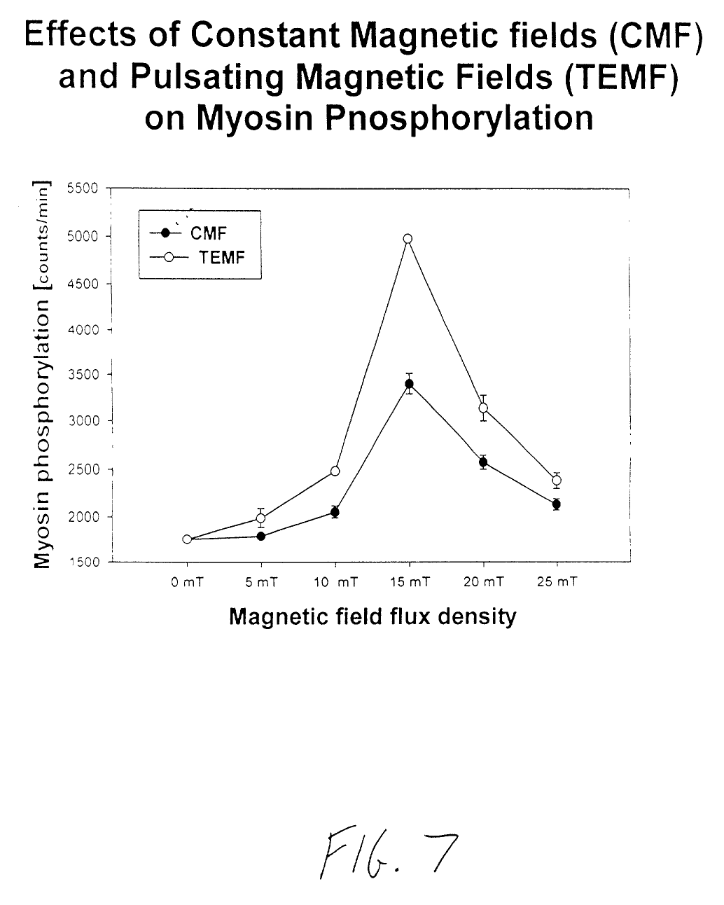

Similarly, the myosin phosphorylation process was also used in studying a pulsating electromagnetic field associated with the EMF Therapeutics Devices. FIG. 7 illustrates by comparison the effects of constant magnetic fields and pulsating magnetic fields on myosin phosphorylation. The pulsating magnetic field associated with the EMF Therapeutic Devices is superior to constant magnetic fields.

Exposure of the myosin phosphorylation model to pulsating magnetic fields in the range of 15-20 mT shows a statistically significant (p<0.05) increase in myosin phosphorylation for all three amplitudes of the applied magnetic field. The strongest increase (94%) was observed for the 15 mT magnetic flux density while for a magnetic flux density of 20 mT the increase was 55%. As mentioned earlier, data was also obtained for the broader range of magnetic flux densities (5-55 mT) (FIG. 6).

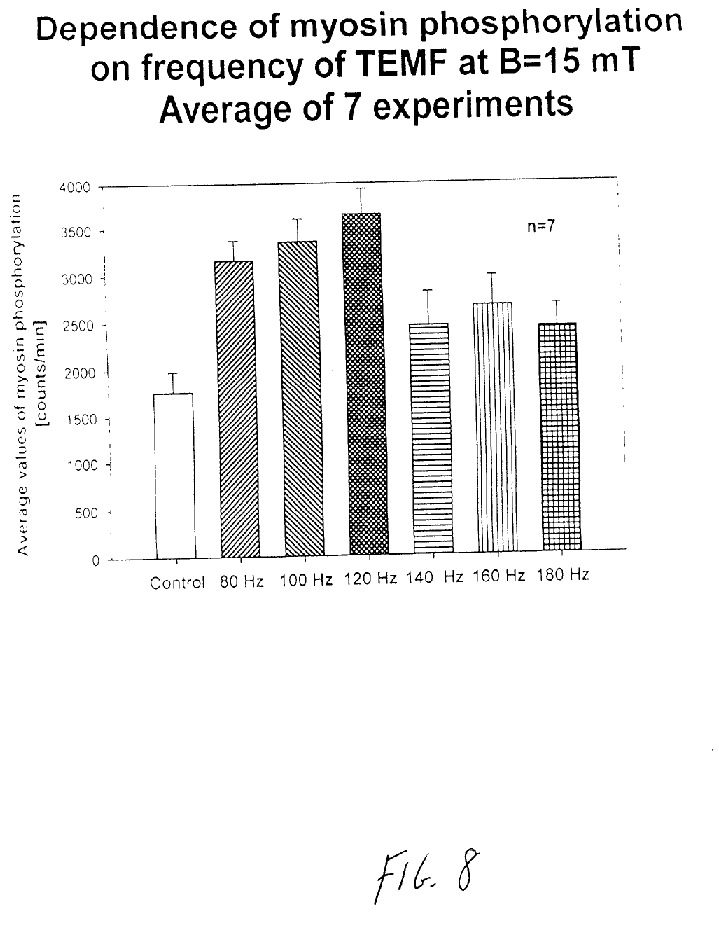

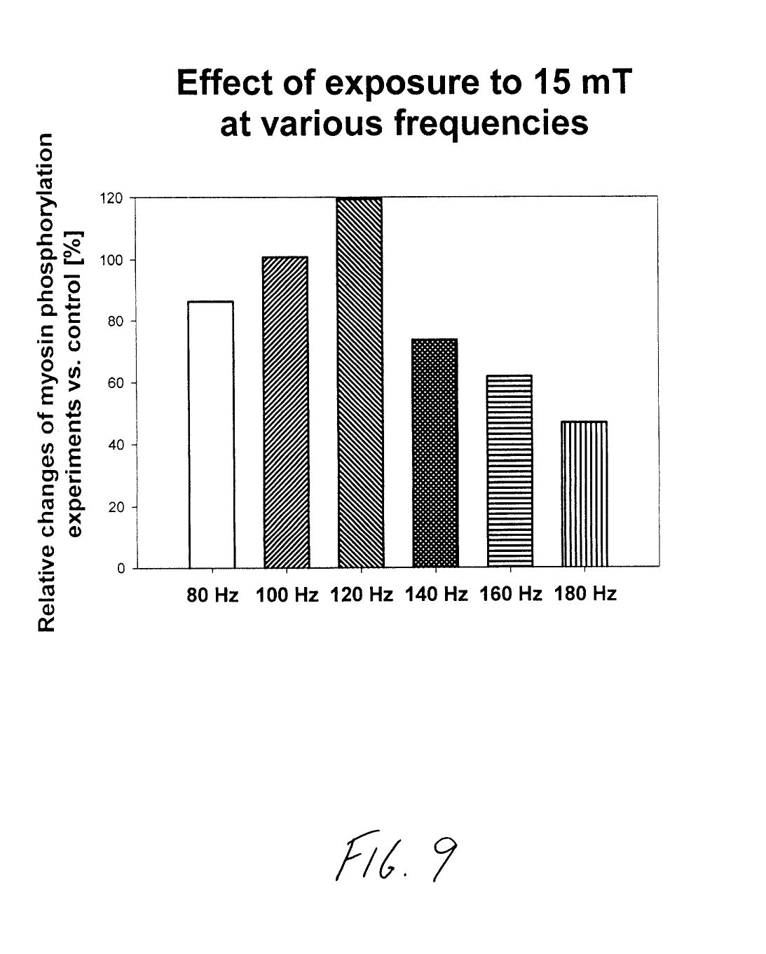

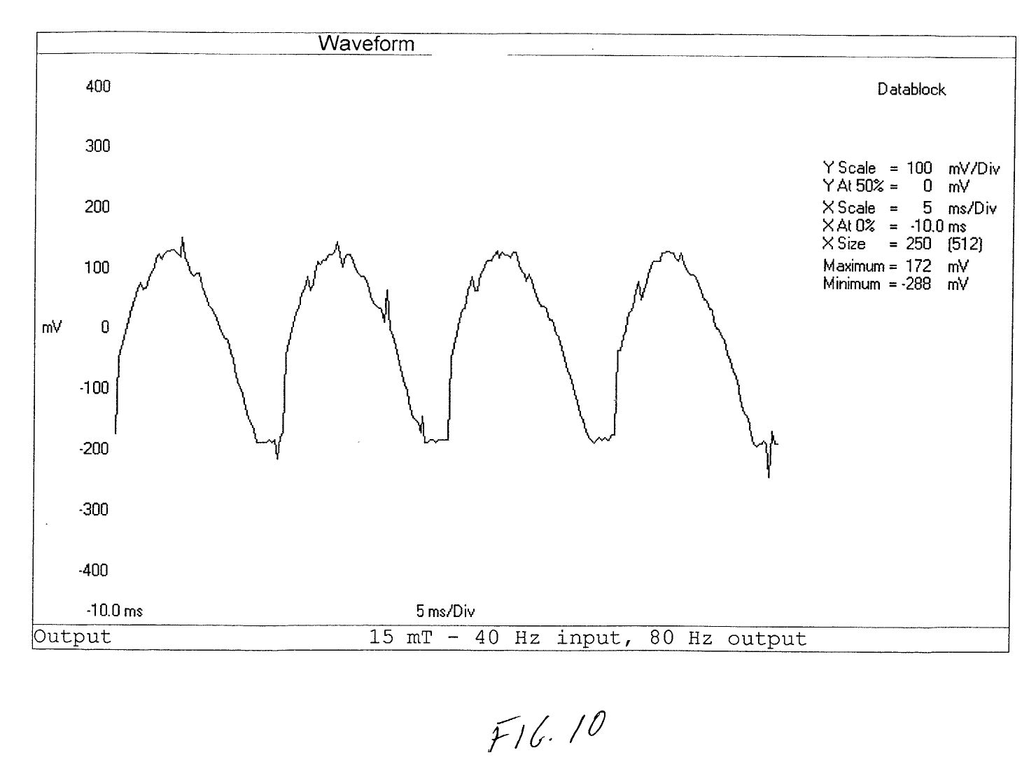

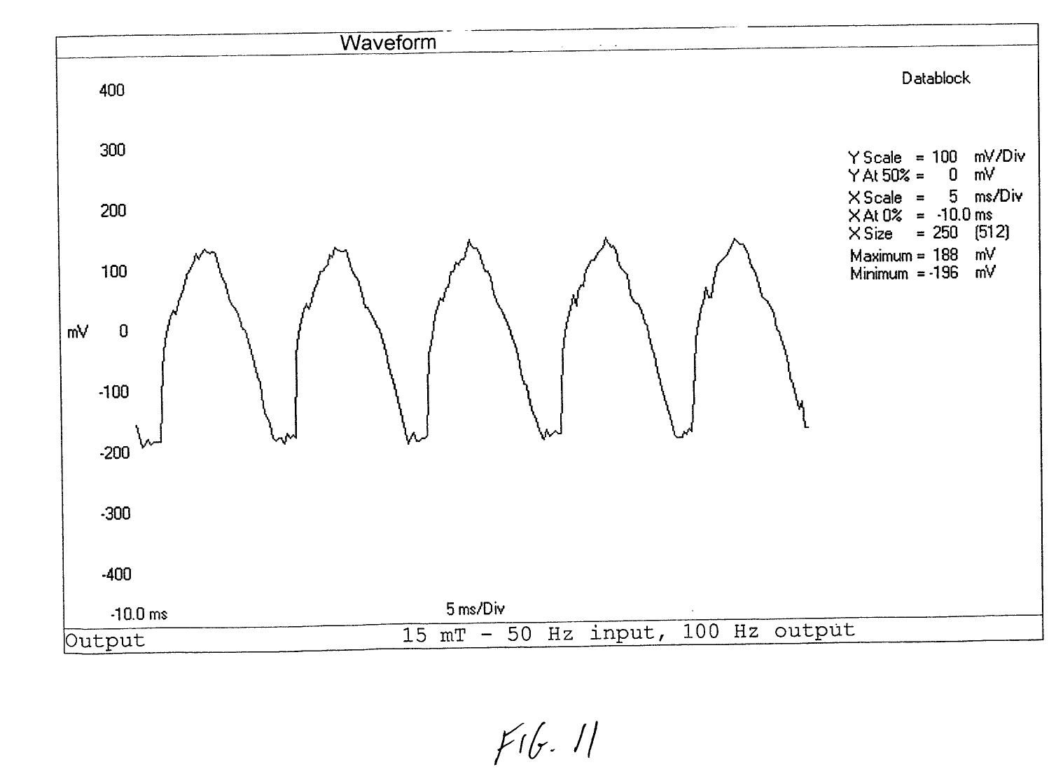

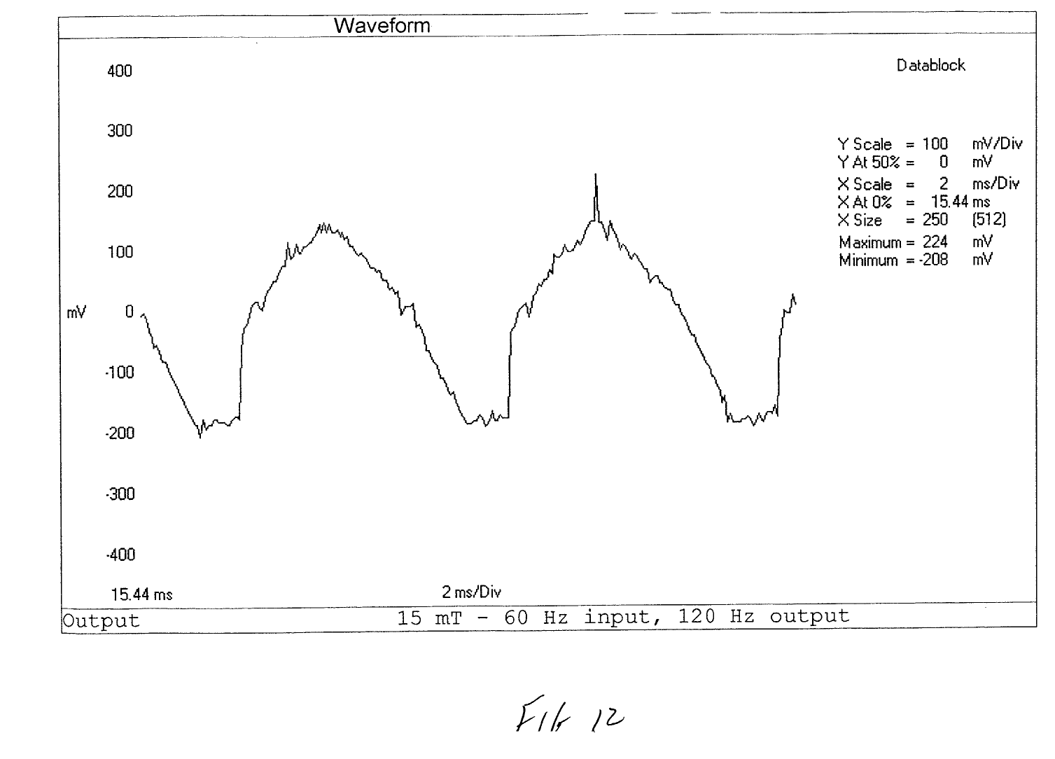

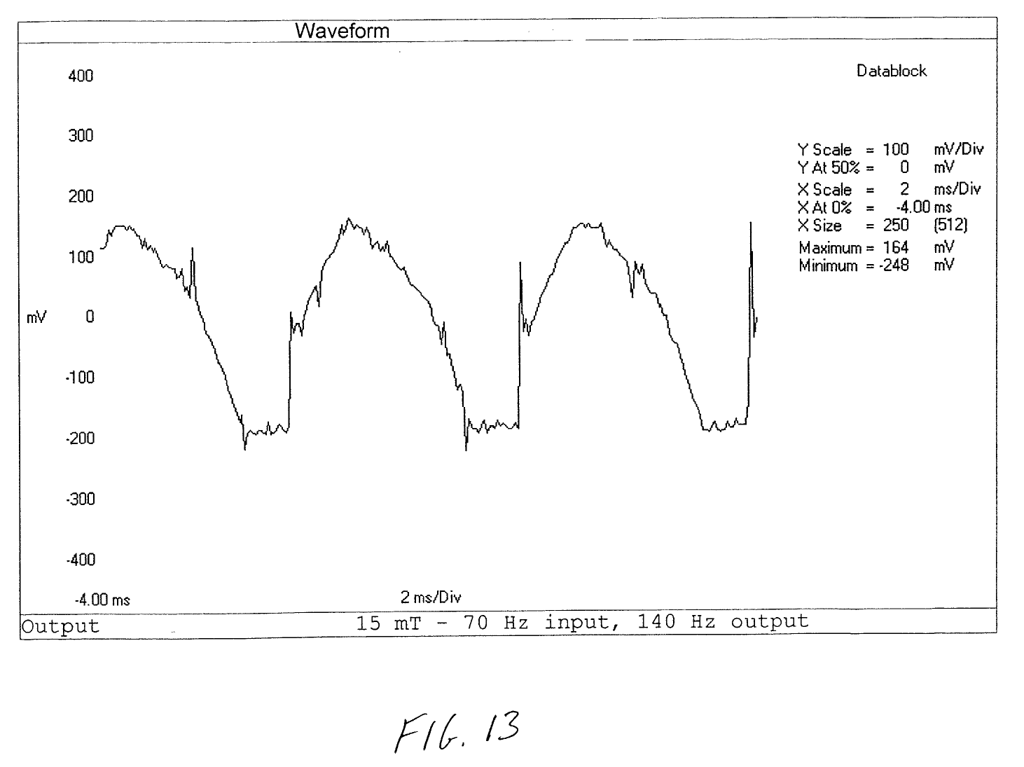

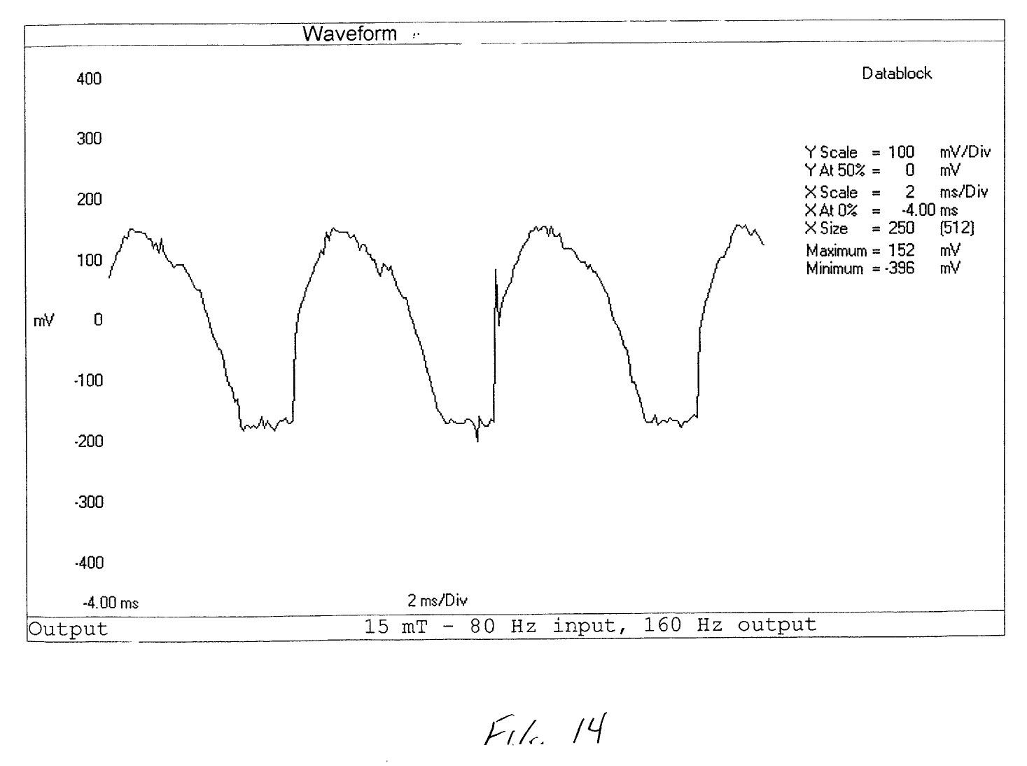

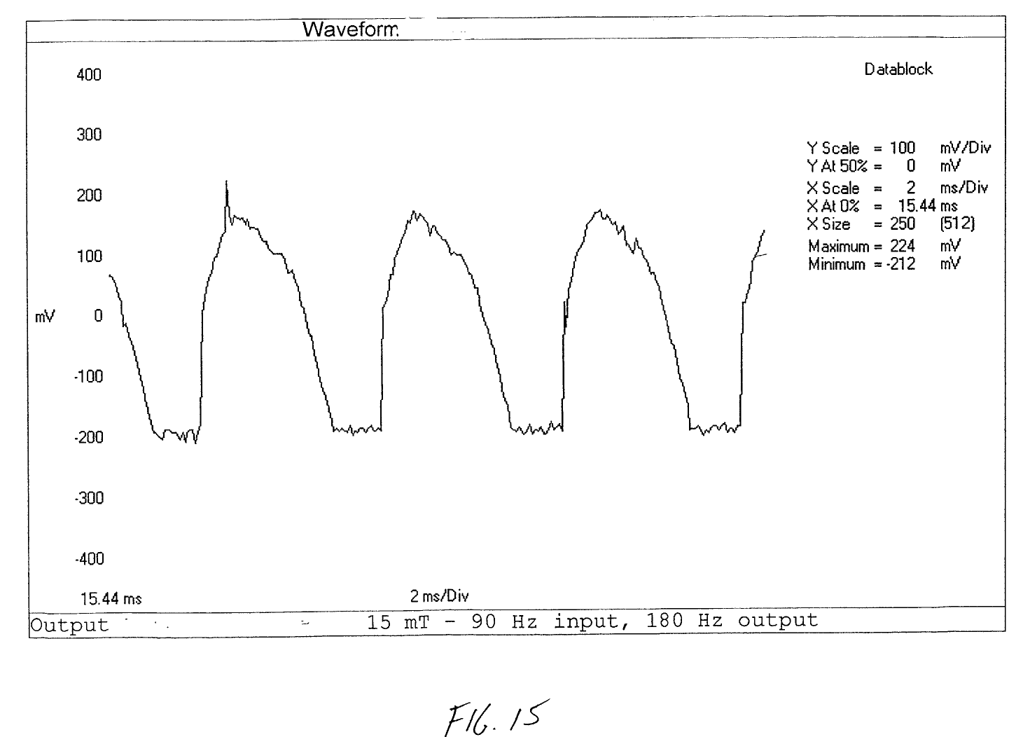

FIGS. 8 and 9 graphically illustrates the effect of exposure to a 15 mT pulsating field from the EMF Therapeutics Devices at various frequencies (see also FIGS. 10-15). The inventive method of using the myosin phosphorylation technique disclosed herein was the tool by which a frequency window was determined to correspond to the known and confirmed amplitude window of 15 mT and the preferred frequency of 120 Hz (pulses per second) as used to obtain the animal data in the “Study 4—EXAMPLE FOUR” of U.S. Pat. No. 6,083,149 mentioned earlier. The graphs of FIGS. 8 and 9 illustrate the existence of the preferred biological frequency window of 120 and 100 pulses per second (“Hz”). The 120 or 100 Hz window is independent of the frequency of the input supply voltage because a 60 Hz input voltage was used for the test as was used in the aforementioned “Study—EXAMPLE FOUR” of U.S. Pat. No. 6,083,149.

Using the myosin phosphorylation technique it is shown that twice the frequencies of commercially available power supply is preferred at 15mT-20mT and 45-50mT amplitude. Thus, the 100 Hz and 120 Hz optimum frequencies form the frequency windows at 15mT-20mT and 45-50mT can be referred to as a 50 Hz “European” preferred frequency window and a 60 Hz “American” preferred frequency window owing to the basic differences between the electric power supplies of Europe and the United States of America. The overall preferred embodiment of the frequency window at 15 mT-20 mT and 45-50 mT is the 120 Hz frequency associated with the American window.

It should also be known, in order to accurately assess the effect of the pulsating magnetic field modulation of cell free myosin phosphorylation, in all experiments a sham-control assay was run (the reaction mixture was placed inside the coil with no current supplying the coil. Therefore, the sham-control samples were exposed only to ambient magnetic fields.

The results of the various experiments indicate that pulsating magnetic fields of 120 Hz (pps) frequency and 15 mT-20 mT and 45-50 mT in amplitude can initiate a biological response in accordance with the “window” hypothesis proving by confirmation the experimental tests conducted on mammals-the data for which is shown in FIGS. 4 and 5 with respect to amplitude and 120 Hz (pps) frequency, and later confirmed by the data associated with FIGS. 8 and 9 with respect to frequency. Hence, the results presented here suggest that this innovative cell-free myosin phosphorylation model can be employed for fast screening of various magnetic field signals.

These and other embodiments of the present inventions are considered to be with the scope of the present invention as claimed below, and all such embodiments and equivalents thereof covered by the scope of those claims even though not specifically set forth herein.

What is claimed is:

1. A method for determining a biological window of a magnetic field comprising the steps of:
preparing a reaction solution containing at least the following components: MLC, MLCK, calmodulin, calcium ions, and radiolabeled ATP, and exposing the reaction solution to a magnetic field;
removing the reaction mixture from the magnetic field and forming a specimen by placing a quantity of the solution onto a substrate;
washing the specimen;
placing the washed specimen in a suspension liquid and counting the number of radioactive events over a given time T.

2. The method of claim 1, wherein exposing the specimen includes the following step:
exposing the specimen for a period of time within the linear portion of the time dependence curve of myosin phosphorylation rate.

3. The method of claim 2, further including the step of:
exposing the specimen to the magnetic field for a period within the range of time between 2 and 6 minutes.

4. The method of claim 3, further including the step of exposing the specimen to the magnetic field for a period of 5 minutes.

5. The method of claim 1, wherein the step of removing the reaction mixture includes the following step:
stopping the reaction by adding a stopping solution to the specimen.

6. The method of claim 1, wherein the step of removing the reaction mixture includes the following step:
stopping the reaction by eliminating the effect of the magnetic field on the specimen.

7. The method of claim 1, wherein the step of placing a quantity of the solution onto a substrate includes the following step:
placing a quantity of solution on filter paper.

8. The method of claim 1, wherein the step of washing the specimen includes the following step:
placing the specimen in a container containing an acidic solvent.

9. The method of claim 8, wherein the step of placing the specimen in a container containing an acidic solvent includes the following step:
placing the specimen in a container containing a solution of TCA.

10. The method of claim 8, wherein the step of placing the specimen in a container containing an acidic solvent includes the following step:
placing the specimen in a container containing an acidic solvent and agitating the solvent.

11. The method of claim 1; wherein the step of placing the washed specimen in a suspension liquid includes the following step:
placing the washed specimen in a suspension of water.

12. The method of claim 11, wherein the step of placing the washed specimen in a suspension of water:
placing the washed specimen in a container of water suitable for use in a liquid scintillation counter.

13. The method of claim 1, wherein the step of counting the number of radioactive events over a given time T includes the following step:
counting the radioactive events by counting the Cherenkov emissions.

14. The method of claim 1, wherein exposing the reaction solution to a magnetic field includes the following step:
creating a magnetic field prior to exposure such that the magnetic field has a frequency of 80 to 180 pulses per second.

15. The method of claim 14, wherein the step of creating a magnetic field prior to exposure such that the magnetic field has a frequency of 80 to 180 pulses per second further includes the following step:
creating a magnetic field having a frequency of between 80 and 140 pulses per second.

16. The method of claim 15, wherein the step of creating a magnetic field prior to exposure such that the magnetic field has a frequency of 80 to 140 pulses per second further includes the following step:
creating a magnetic field having a frequency of between 80 and 120 pulses per second.

17. The method of claim 16, wherein the step of creating a magnetic field prior to exposure such that the magnetic field has a frequency of 80 to 120 pulses per second further includes the following step:
creating a magnetic field having a frequency of between 100 and 120 pulses per second.

18. The method of claim 17, wherein the step of creating a magnetic field prior to exposure such that the magnetic field has a frequency of 100 to 120 pulses per second further includes the following step:
creating a magnetic field having a frequency of 100 pulses per second.

19. The method of claim 17, wherein the step of creating a magnetic field prior to exposure such that the magnetic field has a frequency of 100 to 120 pulses per second further includes the following step:
creating a magnetic field having a frequency of 120 pulses per second.

20. The method of claim 1, wherein exposing the reaction solution to a magnetic field includes the following step:
creating a magnetic field prior to exposure such that the magnetic field has a frequency of 100 to 120 pulses per second.

21. The method of claim 20, wherein the step of creating a magnetic field prior to exposure such that the magnetic field has a frequency of 100 to 120 pulses per second further includes the following step:
creating a magnetic field having a frequency of 100 pulses per second.

22. The method of claim 20, wherein the step of creating a magnetic field prior to exposure such that the magnetic field has a frequency of 100 to 120 pulses per second further includes the following step:
creating a magnetic field having a frequency of 120 pulses per second.

23. The method of claim 1, wherein exposing the reaction solution to a magnetic field includes the following step:
creating a magnetic field prior to exposure such that the magnetic field has an amplitude between 5 and 55 milliTesla.

24. The method of claim 23, wherein the step of creating a magnetic field prior to exposure such that the magnetic field has an amplitude between 5 and 55 milliTesla further includes the following step of:
creating a magnetic field prior to exposure such that the magnetic field has an amplitude between 10 and 25 milliTesla.

25. The method of claim 23, wherein the step of creating a magnetic field prior to exposure such that the magnetic field has an amplitude between 5 and 55 milliTesla further includes the following step of:
creating a magnetic field prior to exposure such that the magnetic field has an amplitude between 40 and 55 milliTesla.

26. The method of claim 24, wherein the step of creating a magnetic field prior to exposure such that the magnetic field has an amplitude between 10 and 25 milliTesla further includes the following step of:
creating a magnetic field prior to exposure such that the magnetic field has an amplitude between 15 and 20 milliTesla.

27. The method of claim 25, wherein the step of creating a magnetic field prior to exposure such that the magnetic field has an amplitude between 40 and 55 milliTesla further includes the following step of:
creating a magnetic field prior to exposure such that the magnetic field has an amplitude between 45 and 50 milliTesla.

28. The method of claim 26, wherein the step of creating a magnetic field prior to exposure such that the magnetic field has an amplitude between 15 and 20 milliTesla further includes the following step of:
creating a magnetic field prior to exposure such that the magnetic field has an amplitude 17 of 15 milliTesla.

29. The method of claim 27, wherein the step of creating a magnetic field prior to exposure such that the magnetic field has an amplitude between 45 and 50 milliTesla further includes the following step of:
creating a magnetic field prior to exposure such that the magnetic field has an amplitude of 45 milliTesla.

30. The method of claim 1, wherein the step of creating a magnetic field prior to exposure such further includes the following step of creating a magnetic field prior to exposure such that the magnetic field has an amplitude of 15-50 milliTesla.

31. A method for determining a relative biological effectiveness of a magnetic field using cell free myosin phosphorylation comprising the steps of:
preparing a reaction solution containing at least the following components: MLC, MLCK, calmodulin, calcium ions, and radiolabeled ATP, and
exposing a first volume of the reaction solution to a first magnetic field, and
exposing a second volume of the reaction solution to a second magnetic field;
removing the reaction mixture from the first magnetic field and forming a first specimen by placing a quantity of the first volume of solution onto a substrate;
removing the reaction mixture from the second magnetic field and forming a second specimen by placing a quantity of the second volume of solution onto a substrate;
washing the first specimen;
washing the second specimen;
placing the washed first specimen in a suspension and counting the number of radioactive events over a given time T; and
placing the washed second specimen in a suspension and counting the number of radioactive events over a given time T.

32. The method of claim 31, wherein exposing the first specimen and second specimen includes the following step:
exposing the first specimen to the first magnetic field and exposing the second specimen to the second magnetic field both for a period of time within the linear portion of the time dependence curve of myosin phosphorylation rate.

33. The method of claim 32, further including the step of
exposing the first specimen to the first magnetic field for a period within the range of time between 2 and 6 minutes, and
exposing the second specimen to the second magnetic field for a period within the range of time between 2 and 6 minutes.

34. The method of claim 33, further including the step of
exposing the first specimen to the first magnetic field for a period of 5 minutes; and
exposing the second specimen to the second magnetic field for a period of 5 minutes.

35. The method of claim 31, wherein exposing the first specimen and exposing the second specimen both include the following step of:
stopping the reaction by adding a stopping solution to the specimen.

36. The method of claim 31, wherein exposing the first specimen and exposing the second specimen includes the following step:
stopping the reaction by eliminating the effect of the first magnetic field on the first specimen; and
stopping the reaction by eliminating the effect of the second magnetic field on the second specimen.

37. The method of claim 31, wherein the step of placing a quantity of the first volume and second volume of the solution onto a substrate includes the following step:
placing a quantity of the first volume of solution on filter paper; and
placing a quantity of the second volume of solution on filter paper.

38. The method of claim 31, wherein the step of washing the first specimen and second specimen includes the following step:
placing the first specimen in a container containing an acidic solvent; and
placing the second specimen in a container containing an acidic solvent.

39. The method of claim 38, wherein the step of placing the first and second specimen in a container containing an acidic solvent includes the following step:
placing the first specimen in a container containing a solution of TCA; and
placing the second specimen in a container containing a solution of TCA.

40. The method of claim 38, wherein the step of placing the first and second specimen in a container containing an acidic solvent includes the following step:
placing the first specimen in a container containing an acidic solvent and agitating the solvent; and
placing the second specimen in a container containing an acidic solvent and agitating the solvent.

41. The method of claim 31, wherein the step of placing the washed first specimen and washed second specimen in a suspension liquid includes the following step:
placing the washed first specimen in a suspension of water; and
placing the washed second specimen in a suspension of water.

42. The method of claim 41, wherein the step of placing the washed first specimen and washed second specimen in a suspension of water:
placing the washed first specimen in a container of water suitable for use in a liquid scintillation counter; and
placing the washed second specimen in a container of water suitable for use in a liquid scintillation counter.

43. The method of claim 31, wherein the step of counting the number of radioactive events of either the first or second specimen over a given time T includes the following step:
counting the radioactive events by counting the Cherenkov emissions.

44. The method of claim 31, wherein exposing the reaction solution to a first or second magnetic field includes the following step:
creating a first or second magnetic field prior to exposure such that the first or second 27 magnetic field has a frequency of 80 to 180 pulses per second.

45. The method of claim 44, wherein the step of creating a first or second magnetic field prior to exposure such that the first or second magnetic field has a frequency of 80 to 180 pulses per second further includes the following step:
creating a first or second magnetic field having a frequency of between 80 and 140 pulses per second.

46. The method of claim 45, wherein the step of creating a first or second magnetic field prior to exposure such that the first or second magnetic field has a frequency of 80 to 140 pulses per second further includes the following step:
creating a first or second magnetic field having a frequency of between 80 and 120 pulses per second.

47. The method of claim 46, wherein the step of creating a first or second magnetic field prior to exposure such that the first or second magnetic field has a frequency of 80 to 120 pulses per second further includes the following step:
creating a first or second magnetic field having a frequency of between 100 and 120 pulses per second.

48. The method of claim 47, wherein the step of creating a first or second magnetic field prior to exposure such that the first or second magnetic field has a frequency of 100 to 120 pulses per second further includes the following step:
creating a first or second magnetic field having a frequency of 100 pulses per second.

49. The method of claim 47, wherein the step of creating a first or second magnetic field prior to exposure such that the first or second magnetic field has a frequency of 100 to 120 pulses per second further includes the following step:
creating a first or second magnetic field having a frequency of 120 pulses per second.

50. The method of claim 31, wherein exposing the reaction solution to a first or second magnetic field includes the following step:
creating a first or second magnetic field prior to exposure such that the first or second magnetic field has a frequency of 100 to 120 pulses per second.

51. The method of claim 50, wherein the step of creating a first or second magnetic field prior to exposure such that the first or second magnetic field has a frequency of 100 to 120 pulses 8 per second further includes the following step:
creating a first or second magnetic field having a frequency of 100 pulses per second.

52. The method of claim 50, wherein the step of creating a first or second magnetic field prior to exposure such that the first or second magnetic field has a frequency of 100 to 120 pulses per second further includes the following step:
creating a first or second magnetic field having a frequency of 120 pulses per second.

53. The method of claim 31, wherein exposing the reaction solution to a first or second magnetic field includes the following step:
creating a first or second magnetic field prior to exposure such that the first or second magnetic field has an amplitude between 5 and 55 milliTesla.

54. The method of claim 53, wherein the step of creating a first or second magnetic field prior to exposure such that the first or second magnetic field has an amplitude between 5 and 55 milliTesla further includes the following step of:
creating a first or second magnetic field prior to exposure such that the first or second magnetic field has an amplitude between 10 and 50 milliTesla.

55. The method of claim 54, wherein the step of creating a first or second magnetic field prior to exposure such that the first or second magnetic field has an amplitude between 10 and 50 milliTesla further includes the following step of
creating a first or second magnetic field prior to exposure such that the first or second magnetic field has an amplitude between 15 and 45 milliTesla.

56. The method of claim 54, wherein the step of creating a first or second magnetic field prior to exposure such that the first or second magnetic field has an amplitude between 10 and 50 milliTesla further includes the following step of:
creating a first or second magnetic field prior to exposure such that the first or second magnetic field has an amplitude between 10 and 25 milliTesla.

57. The method of claim 56, wherein the step of creating a first or second magnetic field prior to exposure such that the first or second magnetic field has an amplitude between 10 and 25 milliTesla further includes the following step of:
creating a first or second magnetic field prior to exposure such that the first or second magnetic field has an amplitude between 15 and 20 milliTesla.

58. The method of claim 57, wherein the step of creating a first or second magnetic field prior to exposure such that the first or second magnetic field has an amplitude between 10 and 20 milliTesla further includes the following step of:
creating a first or second magnetic field prior to exposure such that the first or second magnetic field has an amplitude of 15 milliTesla.

59. The method of claim 54, wherein the step of creating a first or second magnetic field prior to exposure such that the first or second magnetic field has an amplitude between 10 and 50 milliTesla further includes the following step of:
creating a first or second magnetic field prior to exposure such that the first or second magnetic field has an amplitude between 40 and 50 milliTesla.

60. The method of claim 59, wherein the step of creating a first or second magnetic field prior to exposure such that the first or second magnetic field has an amplitude between 40 and 50 milliTesla further includes the following step of:
creating a first or second magnetic field prior to exposure such that the first or second magnetic field has an amplitude of 45 milliTesla.

61. The method of claim 31 wherein the step of counting a number of radioactive events associated with the first specimen over a time T, and the step of counting a number of radioactive events associated with the second specimen over a time T further includes the step of:
comparing the number of radioactive events associated with the first specimen to the number of radioactive events associated with the second specimen.

62. An apparatus for generating a magnetic field found to coincide with a biological window of magnetic field metrics, comprising:
a coil assembly including at least one electrical conductor; and
a source of electric current applied to the length of electrical conductor to create a magnetic field having an amplitude within a known biological magnetic field metric window within the interior of the coil.

63. The apparatus of claim 62, further comprising:
a frame defining a coil assembly interior when the coil is wrapped about the frame.

64. The apparatus of claim 63, further comprising:
a central passageway extending through the frame; and
a useful magnetic field frequency in pulses per second which is double the frequency of the input current obtained from a readily available commercial electric power supply.

65. The apparatus of claim 64, further comprising:
a rectifier for doubling the frequency of the input electric current.

66. The apparatus of claim 62, wherein:
the coil assembly is configured to create a magnetic field having a frequency of 80 to 180 pulses per second.

67. The apparatus of claim 66, wherein:
the coil assembly is configured to create a magnetic field having a frequency of 80 to 140 pulses per second.

68. The apparatus of claim 67, wherein:
the coil assembly is configured to create a magnetic field having a frequency of 80 to 120 pulses per second.

69. The apparatus of claim 68, wherein:
the coil assembly is configured to create a magnetic field having a frequency of 100 to 120 pulses per second.

70. The apparatus of claim 69, wherein:
the coil assembly is configured to create a magnetic field having a frequency of 100 pulses per second.

71. The apparatus of claim 69, wherein:
the coil assembly is configured to create a magnetic field having a frequency of 120 pulses per second.

72. The apparatus of claim 62, wherein:
the coil assembly is configured to create a magnetic field having a frequency of 100 to 120 pulses per second.

73. The apparatus of claim 72, wherein:
the coil assembly is configured to create a magnetic field having a frequency of 100 pulses per second.

74. The apparatus of claim 72, wherein:
the coil assembly is configured to create a magnetic field having a frequency of 120 pulses per second.

75. The apparatus of claim 62, wherein:
the coil assembly is configured to create a magnetic field having an amplitude between 5 and 55 milliTesla.

76. The apparatus of claim 75, wherein:
the coil assembly is configured to create a magnetic field having an amplitude between 10 and 50 milliTesla.

77. The apparatus of claim 76, wherein:
the coil assembly is configured to create a magnetic field having an amplitude between 15 and 45 milliTesla.

78. The apparatus of claim 77, wherein:
the coil assembly is configured to create a magnetic field having an amplitude between 10 and 25 milliTesla.

79. The apparatus of claim 78, wherein:
the coil assembly is configured to create a magnetic field having an amplitude between 15 and 20 milliTesla.

80. The apparatus of claim 79, wherein:
the coil assembly is configured to create a magnetic field having an amplitude of 15 milliTesla.

81. The apparatus of claim 75, wherein:
the coil assembly is configured to create a magnetic field having an amplitude between 40 and 50 milliTesla.

82. The apparatus of claim 81, wherein:
the coil assembly is configured to create a magnetic field having an amplitude of 45 milliTesla.
